(19) United States
(12) Patent Application Publication
(10) Pub. No.: US 2022/0275093 A1
(43) Pub. Date: Sep. 1, 2022

(54) TREATMENT OF CANCER USING A HLA-A2/WT1 X CD3 BISPECIFIC ANTIBODY AND LENALIDOMIDE

(71) Applicant: Hoffmann-La Roche Inc., Little Falls, NJ (US)

(72) Inventors: Christian AUGSBERGER, Germering (DE); Christian KLEIN, Weilheim (DE); Sabine Marion SUBKLEWE, Muenchen (DE)

(73) Assignee: Hoffmann-La Roche Inc., Little Falls, NJ (US)

(21) Appl. No.: 17/662,117

(22) Filed: May 5, 2022

Related U.S. Application Data
(63) Continuation of application No. PCT/EP2020/080763, filed on Nov. 3, 2020.

(30) Foreign Application Priority Data
Nov. 5, 2019 (EP) 19207090.2

(51) Int. Cl. C07K 16/28; A61K 31/445; A61P 35/00; C07K 16/32; C07K 16/46

(52) U.S. Cl. CPC C07K 16/2833; A61K 31/445; A61P 35/00; C07K 16/2809; C07K 16/32; C07K 16/468; C07K 2317/31; C07K 2317/64

(57) ABSTRACT

The present invention relates to the treatment of cancer, in particular to the treatment of cancer using a HLA-A2/WT1×CD3 bispecific antibody and lenalidomide.

### FIELD OF THE INVENTION

The present invention relates to the treatment of cancer, in particular to the treatment of cancer using a HLA-A2/WT1×CD3 bispecific antibody and lenalidomide.

### BACKGROUND

T-cell activating bispecific antibodies are a novel class of cancer therapeutics, designed to engage cytotoxic T cells against tumor cells. The simultaneous binding of such an antibody to CD3 on T-cells and to an antigen expressed on the tumor cells will force a temporary interaction between tumor cell and T cell, causing activation of the T-cell and subsequent lysis of the tumor cell.

WT1 (Wilms tumor 1, Wilms tumor protein) is an oncogenic transcription factor involved in cell proliferation, differentiation, as well as apoptosis and organ development, whose expression in normal adult tissue is rare (Hinrichs and Restifo, Nat Biotechnol (2013) 31, 999-1008). WT1 is, however, reported to be overexpressed in several types of haematological maligancies and a wide range of solid tumors (Van Driessche et al., Oncologist (2012) 17, 250-259). WT1 is a nuclear protein, localized intracellularly. Intracellular protein can be degraded in the proteasome, processed and presented on the cell surface by major histocompatibility complex (MHC) I as T cell epitopes, and recognized by T cell receptors (TCR). As such, WT1-derived peptides are presented in the context of HLA-A2 on the cell surface and can trigger T cell recognition.

T-cell activating bispecific antibodies targeting HLA-A2/WT1 have been described in WO 2019/122052. Such T-cell activating bispecific antibodies may be useful, e.g., in the treatment of acute myeloid leukemia (AML).

In order to maximize the therapeutic benefit of HLA-A2/WT1-targeting T-cell activating antibodies, e.g. in AML, it would thus be desirable to identify combination treatments involving such T-cell activating antibodies and other therapeutic agents.

### DESCRIPTION OF THE INVENTION

The present inventors have found that combination of HLA-A2/WT1 targeted T-cell activating bispecific antibodies with lenalidomide leads to enhanced activity in AML as compared to HLA-A2/WT1 targeted T-cell activated bispecific antibody alone.

Using primary AML cells, the inventors have surprisingly found that tumor cell lysis induced by HLA-A2/WT1×CD3 bispecific antibody was enhanced by the addition of lenalidomide.

Accordingly, in a first aspect, the present invention provides a HLA-A2/WT1×CD3 bispecific antibody for use in the treatment of a cancer in an individual, wherein the treatment comprises administration of the HLA-A2/WT1×CD3 bispecific antibody in combination with lenalidomide.

In a further aspect, the invention provides the use of a HLA-A2/WT1×CD3 bispecific antibody in the manufacture of a medicament for the treatment of cancer in an individual, wherein the treatment comprises administration of the HLA-A2/WT1×CD3 bispecific antibody in combination with lenalidomide.

In still a further aspect, the invention provides a method for treating cancer in an individual comprising administering to the individual a HLA-A2/WT1×CD3 bispecific antibody and lenalidomide.

In one aspect, the invention also provides a kit comprising a first medicament comprising a HLA-A2/WT1×CD3 bispecific antibody and a second medicament comprising lenalidomide, and optionally further comprising a package insert comprising instructions for administration of the first medicament in combination with the second medicament for treating cancer in an individual. The HLA-A2/WT1×CD3 bispecific antibodies, methods, uses or kits described above and herein, may incorporate, singly or in combination, any of the features described in the following (unless the context dictates otherwise).

The HLA-A2/WT1×CD3 bispecific antibody herein is a bispecific antibody that specifically binds to CD3 and to HLA-A2/WT1, particularly HLA-A2/WT1_{RMF}. Particularly useful HLA-A2/WT1×CD3 bispecific antibodies are described e.g. in PCT publication no. WO 2019/122052 (incorporated herein by reference in its entirety).

The term “bispecific” means that the antibody is able to specifically bind to at least two distinct antigenic determinants. Typically, a bispecific antibody comprises two antigen binding sites, each of which is specific for a different antigenic determinant. In certain aspects, the bispecific antibody is capable of simultaneously binding two antigenic determinants, particularly two antigenic determinants expressed on two distinct cells.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that specifically binds to an antigenic determinant. In one aspect, an antigen binding moiety is able to direct the entity to which it is attached (e.g. a second antigen binding moiety) to a target site, for example to a specific type of tumor cell bearing the antigenic determinant. In another aspect an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain aspects, the antigen binding moieties may comprise antibody constant regions as further defined herein and known in the art. Useful heavy chain constant regions include any of the five isotypes: α, δ, ϵ, γ, or μ. Useful light chain constant regions include any of the two isotypes: κ and λ.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance

(SPR) technique (analyzed e.g. on a BIAcore instrument) (Liljeblad et al., Glyco J 17, 323-329 (2000)), and traditional binding assays (Heeley, Endocr Res 28, 217-229 (2002)). In one aspect, the extent of binding of an antigen binding moiety to an unrelated protein is less than about 10% of the binding of the antigen binding moiety to the antigen as measured, e.g., by SPR. In certain aspects, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸M to 10⁻¹³M, e.g., from 10⁻⁹M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one aspect, CD3 is human CD3, particularly the epsilon subunit of human CD3 (CD3c). The amino acid sequence of human CD3c is shown in UniProt (www.uniprot.org) accession no. P07766 (version 144), or NCBI (www.ncbi.nlm.nih.gov/) RefSeq NP_000724.1. See also SEQ ID NO: 27. The amino acid sequence of cynomolgus [Macaca fascicularis] CD3_{ϵ} is shown in NCBI GenBank no. BAB71849.1. See also SEQ ID NO: 28.

“WT1”, also known as “Wilms tumor 1” or “Wilms tumor protein”, refers to any native WT1 from any vertebrate source, including mammals such as primates (e.g. humans), non-human primates (e.g. cynomolgus monkeys) and rodents (e.g. mice and rats), unless otherwise indicated. The term encompasses “full-length,” unprocessed WT1 as well as any form of WT1 that results from processing in the cell. The term also encompasses naturally occurring variants of WT1, e.g., splice variants or allelic variants. In one aspect, WT1 is human WT1, particularly the protein of SEQ ID NO: 23. Human WT1 is described in UniProt (www.uniprot.org) accession no. P19544 (entry version 215), and an amino acid sequence of human WT1 is also shown in SEQ ID NO: 23.

By “VLD”, “VLD peptide” or “WT1_{VLD}” is meant the WT1 derived peptide having the amino acid sequence VLDFAPPGA (SEQ ID NO: 24; position 37-45 of the WT1 protein of SEQ ID NO: 23).

By “RMF”, “RMF peptide” or “WT1_{RMF}” is meant the WT1 derived peptide having the amino acid sequence RMFRNAPYL (SEQ ID NO: 25; position 126-134 of the WT1 protein of SEQ ID NO: 23).

“HLA-A2”, “HLA-A*02”, “HLA-A02”, or “HLA-A*2” (used interchangeably) refers to a human leukocyte antigen serotype in the HLA-A serotype group. The HLA-A2 protein (encoded by the respective HLA gene) constitutes the a chain of the respective class I MHC (major histocompatibility complex) protein, which further comprises a β2 microglobulin subunit. A specific HLA-A2 protein is HLA-A201 (also referred to as HLA-A0201, HLA-A02.01, or HLA-A*02:01). In specific aspects, the HLA-A2 protein described herein is HLA-A201. An exemplary sequence of human HLA-A2 is given in SEQ ID NO: 26.

“HLA-A2/WT1” refers to a complex of a HLA-A2 molecule and a WT1 derived peptide (also referred to herein as a “WT1 peptide”), specifically the RMF or VLD peptide (“HLA-A2/WT1_{RMF}” and “HLA-A2/WT1_{VLD}”, respectively). The bispecific antibody used in the present invention specifically may bind to either the HLA-A2/WT1_{RMF} or the HLA-A2/WT1_{VLD} complex.

As used herein, the terms “first”, “second” or “third” with respect to Fab molecules etc., are used for convenience of distinguishing when there is more than one of each type of moiety. Use of these terms is not intended to confer a specific order or orientation of the bispecific antibody unless explicitly so stated.

The term “valent” as used herein denotes the presence of a specified number of antigen binding sites in an antibody. As such, the term “monovalent binding to an antigen” denotes the presence of one (and not more than one) antigen binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, Md. (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, Md. (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)). Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1 (L1)-FR2-H2(L2)-FR3-H3 (L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ϵ, γ, and μ, and respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover

Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ϵ (IgE), γ (IgG), or μ (IgM), some of which may be further divided into subtypes, e.g. γ₁ (IgG1), γ₂ (IgG2), γ₃ (IgG3), γ₄ (IgG4), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain.

However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some aspects the modification promoting association comprises an amino acid mutation in the Fc domain, specifically an amino acid substitution. In a particular aspect, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R.

Pearson (1996) “Effective protein sequence comparison” Meth. Enzymol. 266:227- 258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fastawww2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

The HLA-A2/WT1×CD3 bispecific antibody comprises a first antigen binding moiety that specifically binds to CD3, and a second antigen binding moiety that specifically binds to HLA-A2/WT1, particularly HLA-A2/WT1_{RMF}.

In one aspect, the first antigen binding moiety comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6.

In one aspect, the second antigen binding moiety comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

In a particular aspect, the HLA-A2/WT1×CD3 bispecific antibody comprises
(i) a first antigen binding moiety that specifically binds to CD3 and comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6; and
(ii) a second antigen binding moiety that specifically binds to HLA-A2/WT1 and comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

In one aspect, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen binding moiety comprises the heavy chain variable region sequence of SEQ ID NO: 7 and the light chain variable region sequence of SEQ ID NO: 8.

In one aspect, the second antigen binding moiety comprises a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one aspect, the second antigen binding moiety comprises the heavy chain variable region sequence of SEQ ID NO: 15 and the light chain variable region sequence of SEQ ID NO: 16.

In some aspects, the first and/or the second antigen binding moiety is a Fab molecule. In some aspects, the first antigen binding moiety is a crossover Fab molecule wherein either the variable or the constant regions of the Fab light chain and the Fab heavy chain are exchanged. In such aspects, the second antigen binding moiety preferably is a conventional Fab molecule.

In some aspects wherein the first and the second antigen binding moiety of the bispecific antibody are both Fab molecules, and in one of the antigen binding moieties (particularly the first antigen binding moiety) the variable domains VL and VH of the Fab light chain and the Fab heavy chain are replaced by each other,
i) in the constant domain CL of the first antigen binding moiety the amino acid at position 124 is substituted by a positively charged amino acid (numbering according to Kabat), and wherein in the constant domain CH1 of the first antigen binding moiety the amino acid at position 147 or the amino acid at position 213 is substituted by a negatively charged amino acid (numbering according to Kabat EU index); or
ii) in the constant domain CL of the second antigen binding moiety the amino acid at position 124 is substituted by a positively charged amino acid (numbering according to Kabat), and wherein in the constant domain CH1 of the second antigen binding moiety the amino acid at position 147 or the amino acid at position 213 is substituted by a negatively charged amino acid (numbering according to Kabat EU index).

The bispecific antibody does not comprise both modifications mentioned under i) and ii). The constant domains CL and CH1 of the antigen binding moiety having the VH/VL exchange are not replaced by each other (i.e. remain unexchanged).

In a more specific aspect,
i) in the constant domain CL of the first antigen binding moiety the amino acid at position 124 is substituted independently by lysine (K), arginine (R) or histidine (H) (numbering according to Kabat), and in the constant domain CH1 of the first antigen binding moiety the amino acid at position 147 or the amino acid at position 213 is substituted independently by glutamic acid (E), or aspartic acid (D) (numbering according to Kabat EU index); or
ii) in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In one such aspect, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In a further aspect, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index).

In preferred aspects, in the constant domain CL of the second antigen binding moiety the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, in the constant domain CL of the second antigen binding moiety the amino acid at position 124 is substituted by lysine (K) (numbering according to Kabat) and the amino acid at position 123 is substituted by lysine (K) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In one aspect, in the constant domain CL of the second antigen binding moiety the amino acid at position 124 is substituted by lysine (K) (numbering according to Kabat) and the amino acid at position 123 is substituted by arginine (R) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In particular aspects, if amino acid substitutions according to the above aspects are made in the constant domain CL and the constant domain CH1 of the second antigen binding moiety, the constant domain CL of the second antigen binding moiety is of kappa isotype.

In some aspects, the first and the second antigen binding moiety are fused to each other, optionally via a peptide linker.

In some aspects, the first and the second antigen binding moiety are each a Fab molecule and either (i) the second antigen binding moiety is fused at the C-terminus of the Fab heavy chain to the N-terminus of the Fab heavy chain of the first antigen binding moiety, or (ii) the first antigen binding moiety is fused at the C-terminus of the Fab heavy chain to the N-terminus of the Fab heavy chain of the second antigen binding moiety.

In some aspects, the HLA-A2/WT1×CD3 bispecific antibody provides monovalent binding to CD3.

In particular aspects, the HLA-A2/WT1×CD3 bispecific antibody comprises a single antigen binding moiety that specifically binds to CD3, and two antigen binding moieties that specifically bind to HLA-A2/WT1. Thus, in some aspects, the HLA-A2/WT1×CD3 bispecific antibody comprises a third antigen binding moiety, particularly a Fab molecule, more particularly a conventional Fab molecule, that specifically binds to HLA-A2/WT1. The third antigen binding moiety may incorporate, singly or in combination, all of the features described hereinabove in relation to the second antigen binding moiety (e.g. the CDR sequences, variable region sequences, and/or amino acid substitutions in the constant regions). In some aspects, the third antigen moiety is identical to the first antigen binding moiety (e.g. is also a conventional Fab molecule and comprises the same amino acid sequences).

In particular aspects, the HLA-A2/WT1×CD3 bispecific antibody further comprises an Fc domain composed of a first and a second subunit. In one aspect, the Fc domain is an IgG Fc domain. In a particular aspect, the Fc domain is an IgG₁ Fc domain. In another aspect the Fc domain is an IgG₄ Fc domain. In a more specific aspect, the Fc domain is an IgG4 Fc domain comprising an amino acid substitution at position 5228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular aspect, the Fc domain is a human Fc domain. In a particularly preferred aspect, the Fc domain is a human IgG₁ Fc domain. An exemplary sequence of a human IgG₁ Fc region is given in SEQ ID NO: 29.

In some aspects wherein the first, the second and, where present, the third antigen binding moiety are each a Fab molecule, (a) either (i) the second antigen binding moiety is fused at the C-terminus of the Fab heavy chain to the N-terminus of the Fab heavy chain of the first antigen binding moiety and the first antigen binding moiety is fused at the C-terminus of the Fab heavy chain to the N-terminus of the first subunit of the Fc domain, or (ii) the first antigen binding moiety is fused at the C-terminus of the Fab heavy chain to the N-terminus of the Fab heavy chain of the second antigen binding moiety and the second antigen binding moiety is fused at the C-terminus of the Fab heavy chain to the N-terminus of the first subunit of the Fc domain; and (b) the third antigen binding moiety, where present, is fused at the C-terminus of the Fab heavy chain to the N-terminus of the second subunit of the Fc domain.

In particular aspects, the Fc domain comprises a modification promoting the association of the first and the second subunit of the Fc domain. The site of most extensive protein-protein interaction between the two subunits of a human IgG Fc domain is in the CH3 domain. Thus, in one aspect said modification is in the CH3 domain of the Fc domain.

In a specific aspect said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some aspects, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In a specific such aspect, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some aspects, the Fc domain comprises one or more amino acid substitution that reduces binding to an Fc receptor and/or effector function.

In a particular aspect the Fc receptor is an Fcγ receptor. In one aspect the Fc receptor is a human Fc receptor. In one aspect the Fc receptor is an activating Fc receptor. In a specific aspect the Fc receptor is an activating human Fcγ receptor, more specifically human FcγRIIIa, FcγRI or FcγRIIa, most specifically human FcγRIIIa. In one aspect the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular aspect, the effector function is ADCC.

Typically, the same one or more amino acid substitution is present in each of the two subunits of the Fc domain. In one aspect, the one or more amino acid substitution reduces the binding affinity of the Fc domain to an Fc receptor. In one aspect, the one or more amino acid substitution reduces the binding affinity of the Fc domain to an Fc receptor by at least 2-fold, at least 5-fold, or at least 10-fold.

In one aspect, the Fc domain comprises an amino acid substitution at a position selected from the group of E233, L234, L235, N297, P331 and P329 (numberings according to Kabat EU index). In a more specific aspect, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In one such aspect, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and

P329G (“P329G LALA”, “PGLALA” or “LALAPG”). Specifically, in preferred aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such aspect, the Fc domain is an IgG₁ Fc domain, particularly a human IgG₁ Fc domain.

In preferred aspects, the HLA-A2/WT1×CD3 bispecific antibody comprises
(i) a first antigen binding moiety that specifically binds to CD3, comprising a heavy chain variable region comprising the heavy chain CDR (HCDR) 1 of SEQ ID NO: 1, the HCDR2 of SEQ ID NO:

2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions, particularly the variable regions, of the Fab light chain and the Fab heavy chain are exchanged;
(ii) a second and a third antigen binding moiety that specifically bind to HLA-A2/WT1, comprising a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14, wherein the second and third antigen binding moiety are each a Fab molecule, particularly a conventional Fab molecule;
(iii) an Fc domain composed of a first and a second subunit,
   wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In one aspect, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen binding moiety comprises the heavy chain variable region sequence of SEQ ID NO: 7 and the light chain variable region sequence of SEQ ID NO: 8.

In one aspect, the second and third antigen binding moiety comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one aspect, the second and third antigen binding moieties comprise the heavy chain variable region of SEQ ID NO: 15 and the light chain variable region of SEQ ID NO: 16.

The Fc domain according to the above aspects may incorporate, singly or in combination, all of the features described hereinabove in relation to Fc domains.

In one aspect, the antigen binding moieties and the Fc region are fused to each other by peptide linkers, particularly by peptide linkers as in SEQ ID NO: 18 and SEQ ID NO: 20.

In one aspect, in the constant domain CL of the second and the third Fab molecule under (ii) the amino acid at position 124 is substituted by lysine (K) (numbering according to Kabat) and the amino acid at position 123 is substituted by lysine (K) or arginine (R), particularly by arginine (R) (numbering according to Kabat), and in the constant domain CH1 of the second and the third Fab molecule under (ii) the amino acid at position 147 is substituted by glutamic acid (E) (numbering according to Kabat EU index) and the amino acid at position 213 is substituted by glutamic acid (E) (numbering according to Kabat EU index).

In one aspect, the HLA-A2/WT1×CD3 bispecific antibody comprises a polypeptide (particularly two polypeptides) comprising a sequence that is at least 80%, 85%, 90%, 95%, 96%, 97%, 98%, or 99% identical to the sequence of SEQ ID NO: 17, a polypeptide comprising a sequence that is at least 80%, 85%, 90%, 95%, 96%, 97%, 98%, or 99% identical to the sequence of SEQ ID NO: 18, a polypeptide comprising a sequence that is at least 80%, 85%, 90%, 95%, 96%, 97%, 98%, or 99% identical to the sequence of SEQ ID NO: 19, and a polypeptide comprising a sequence that is at least 80%, 85%, 90%, 95%, 96%, 97%, 98%, or 99% identical to the sequence of SEQ ID NO: 20.

In one aspect, the HLA-A2/WT1×CD3 bispecific antibody comprises a polypeptide (particularly two polypeptides) comprising the sequence of SEQ ID NO: 17, a polypeptide comprising the sequence of SEQ ID NO: 18, a polypeptide comprising the sequence of SEQ ID NO: 19, and a polypeptide comprising the sequence of SEQ ID NO: 20.

The HLA-A2/WT1×CD3 bispecific antibody herein is used in combination with lenalidomide.

The term “lenalidomide” refers to the compound with the chemical name (RS)-3-(4-amino-1-oxo-1,3-dihydro-2H-isoindol-2-yl)piperidine-2,6-dione, and the following chemical structure:

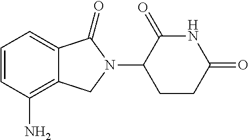 [SMILES: Nc1cccc2c1CN(C1CCC(=O)NC1=O)C2=O]

CAS registry number 191732-72-6. The empirical formula for lenalidomide is C₁₃H₁₃N₃O₃ and the gram molecular weight is 259.3. Lenalidomide is a thalidomide analogue marketed under the tradename REVLIMID®. It is an immunomodulatory agent with anti-angiogenic properties.

Other thalidomide analogues as will be known to the skilled practitioner (e.g. pomalidomide (CAS Registry Number 19171-19-8), avadomide (also known as CC-122; CAS Registry Number 1398053-45-6) or iberdomide (also known as CC-220; CAS Registry Number 1323403-33-3)) are also contemplated for use in the present invention.

The term “cancer” refers to the physiological condition in mammals that is typically characterized by unregulated cell proliferation. Examples of cancer include but are not limited to, carcinoma, lymphoma, blastoma, sarcoma and leukemia. More non-limiting examples of cancers include haematological cancer such as leukemia, bladder cancer, brain cancer, head and neck cancer, pancreatic cancer, biliary cancer, thyroid cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, sarcoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

In some aspects of the HLA-A2/WT1×CD3 bispecific antibodies, methods, uses and kits of the invention, the cancer is a haematological cancer. Non-limiting examples of haematological cancers include leukemia (e.g. acute lymphocytic leukemia (ALL), acute myeloid leukemia (AML), chronic lymphcytic leukemia (CLL) chronic myeloid leukemia (CML), hairy cell leukemia (HCL)), lymphoma (e.g. Non-Hodgkin lymphoma (NHL), Hodgkin lymphoma), myeloma (e.g. multiple myeloma (MM)), myelodysplastic syndrome (MDS) and myeloproliferative diseases.

In certain aspects the cancer is chosen from the group consisting of haematological cancer (such as leukemia), kidney cancer, bladder cancer, skin cancer, lung cancer, colorectal cancer, breast cancer, brain cancer, head and neck cancer and prostate cancer.

In particular aspects, the cancer is a haematological cancer, particularly leukemia, most particularly acute lymphocytic leukemia (ALL) or acute myeloid leukemia (AML).

In preferred aspects the cancer is acute myeloid leukemia (AML).

In further particular aspects, the cancer is myelodysplastic syndrome (MDS).

In some aspects, the cancer is a WT1-positive cancer. By “WT1-positive cancer” or “WT1-expressing cancer” is meant a cancer characterized by expression or overexpression of WT1 in cancer cells. The expression of WT1 may be determined for example by quantitative real-time PCR (measuring WT1 mRNA levels), immunohistochemistry (IHC) or western blot assays. In one aspect, the cancer expresses WT1. In one aspect, the cancer expresses WT1 in at least 20%, preferably at least 50% or at least 80% of tumor cells as determined by immunohistochemistry (IHC) using an antibody specific for WT1.

A “patient”, “subject” or “individual” herein is any single human subject eligible for treatment who is experiencing or has experienced one or more signs, symptoms, or other indicators of cancer. In some aspects, the patient has cancer or has been diagnosed with cancer. The patient may have been previously treated with a HLA-A2/WT1×CD3 bispecific antibody or another drug, or not so treated. In particular aspects, the patient has not been previously treated with a HLA-A2/WT1×CD3 bispecific antibody. The patient may have been treated with a therapy comprising one or more drugs other than a HLA-A2/WT1×CD3 bispecific antibody before the HLA-A2/WT1×CD3 bispecific antibody therapy is commenced.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The HLA-A2/WT1×CD3 bispecific antibody and lenalidomide are administered in an effective amount.

An “effective amount” of an agent, e.g. a pharmaceutical composition, refers to an amount effective, at dosages and for periods of time necessary, to achieve the desired therapeutic or prophylactic result.

In one aspect, administration of the HLA-A2/WT1×CD3 bispecific antibody results in activation of T cells, particularly cytotoxic T cells, particularly at the site of the cancer. Said activation may comprise proliferation of T cells, differentiation of T cells, cytokine secretion by T cells, cytotoxic effector molecule release from T cells, cytotoxic activity of T cells, and expression of activation markers by T cells. In one aspect, the administration of the HLA-A2/WT1×CD3 bispecific antibody results in an increase of T cell, particularly cytotoxic T cell, numbers at the site of the cancer.

In some aspects of the HLA-A2/WT1×CD3 bispecific antibodies, methods, uses or kits described above and herein, the treatment with or administration of the HLA-A2/WT1×CD3 bispecific antibody and lenalidomide results in increased activation of T cells, particularly cytotoxic T cells, particularly at the site of the cancer, as compared to treatment with or administration of the HLA-A2/WT1×CD3 bispecific antibody alone. In particular aspects, the activation comprises cytotoxic activity (specifically lysis of cancer cells) of T cells and/or cytokine (specifically IL-2, TNF-α, and/or interferon-γ) secretion by T cells.

In some aspects of the HLA-A2/WT1×CD3 bispecific antibodies, methods, uses or kits described above and herein, the treatment with or administration of the HLA-A2/WT1×CD3 bispecific antibody and lenalidomide results in increased differentiation of naïve T cells towards memory T cells, particularly at the site of the cancer, as compared to treatment with or administration of the

HLA-A2/WT1×CD3 bispecific antibody alone. In one aspect, the differentiation is detected by measurement of CD45RA expression, e.g. using flow cytometry.

In some aspects of the HLA-A2/WT1×CD3 bispecific antibodies, methods, uses or kits described above and herein, the treatment with or administration of the HLA-A2/WT1×CD3 bispecific antibody and lenalidomide may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the treatment with or administration of the HLA-A2/WT1×CD3 bispecific antibody and lenalidomide may improve the response as compared to treatment with or administration of the HLA-A2/WT1×CD3 bispecific antibody alone (i.e. without lenalidomide).

In some aspects, the treatment or administration of the HLA-A2/WT1×CD3 bispecific antibody and lenalidomide may increase response rates in a patient population, as compared to a corresponding patient population treated with the HLA-A2/WT1×CD3 bispecific antibody alone (i.e. without lenalidomide).

The combination therapy of the invention comprises administration of a HLA-A2/WT1×CD3 bispecific antibody and lenalidomide.

As used herein, “combination” (and grammatical variations thereof such as “combine” or “combining”) encompasses combinations of a HLA-A2/WT1×CD3 bispecific antibody and lenalidomide according to the invention wherein the HLA-A2/WT1×CD3 bispecific antibody and lenalidomide are in the same or in different containers, in the same or in different pharmaceutical formulations, administered together or separately, administered simultaneously or sequentially, in any order, and administered by the same or by different routes, provided that the HLA-A2/WT1×CD3 bispecific antibody and lenalidomide can simultaneously exert their biological effects in the body. For example “combining” HLA-A2/WT1×CD3 bispecific antibody and lenalidomide according to the invention may mean first administering the HLA-A2/WT1×CD3 bispecific antibody in a particular pharmaceutical formulation, followed by administration of lenalidomide in another pharmaceutical formulation, or vice versa.

The HLA-A2/WT1×CD3 bispecific antibody and lenalidomide may be administered in any suitable manner known in the art. In one aspect, the HLA-A2/WT1×CD3 bispecific antibody and lenalidomide are administered sequentially (at different times). In another aspect, the HLA-A2/WT1×CD3 bispecific antibody and lenalidomide are administered concurrently (at the same time). Without wishing to be bound by theory, it may be advantageous to administer lenalidomide prior to and/or concurrently with the HLA-A2/WT1×CD3 bispecific antibody. In some aspects, the HLA-A2/WT1×CD3 bispecific antibody is in a separate composition as lenalidomide. In some aspects, the HLA-A2/WT1×CD3 bispecific antibody is in the same composition as lenalidomide.

The HLA-A2/WT1×CD3 bispecific antibody and lenalidomide can be administered by any suitable route, and may be administered by the same route of administration or by different routes of administration. In some aspects, the HLA-A2/WT1×CD3 bispecific antibody is administered intravenously, intramuscularly, subcutaneously, topically, orally, transdermally, intraperitoneally, intraorbitally, by implantation, by inhalation, intrathecally, intraventricularly, or intranasally. In a particular aspect, the HLA-A2/WT1×CD3 bispecific antibody is administered intravenously. In some aspects, lenalidomide is administered intravenously, intramuscularly, subcutaneously, topically, orally, transdermally, intraperitoneally, intraorbitally, by implantation, by inhalation, intrathecally, intraventricularly, or intranasally. In a particular aspect, lenalidomide is administered orally. An effective amount of the HLA-A2/WT1×CD3 bispecific antibody and lenalidomide may be administered for prevention or treatment of disease. The appropriate route of administration and dosage of the HLA-A2/WT1×CD3 bispecific antibody and/or lenalidomide may be determined based on the type of disease to be treated, the type of the HLA-A2/WT1×CD3 bispecific antibody, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. The HLA-A2/WT1×CD3 bispecific antibody and lenalidomide are suitably administered to the patient at one time or over a series of treatments.

Combinations of the invention can be used either alone or together with other agents in a therapy. For instance, a combination of the invention may be co-administered with at least one additional therapeutic agent. In certain aspects, an additional therapeutic agent is an anti-cancer agent, e.g. a chemotherapeutic agent, an inhibitor of tumor cell proliferation, or an activator of tumor cell apoptosis. Combinations of the invention can also be combined with radiation therapy.

A kit as provided herein typically comprises one or more container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a HLA-A2/WT1×CD3 bispecific antibody to be used in the combinations of the invention. Another active agent is lenalidomide to be used in the combinations of the invention, which may be in the same composition and container like the bispecific antibody, or may be provided in a different composition and container. The label or package insert indicates that the composition(s) is/are used for treating the condition of choice, such as cancer.

In one aspect the invention provides a kit intended for the treatment of cancer, comprising in the same or in separate containers (a) a HLA-A2/WT1×CD3 bispecific antibody, and (b) lenalidomide, and optionally further comprising (c) a package insert comprising printed instructions directing the use of the combined treatment as a method for treating cancer. Moreover, the kit may comprise (a) a first container with a composition contained therein, wherein the composition comprises a HLA-A2/WT1×CD3 bispecific antibody; (b) a second container with a composition contained therein, wherein the composition comprises lenalidomide; and optionally (c) a third container with a composition contained therein, wherein the composition comprises a further cytotoxic or otherwise therapeutic agent. The kit in these aspects of the invention may further comprise a package insert indicating that the compositions can be used to treat cancer. Alternatively, or additionally, the kit may further comprise a third (or fourth) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Amino Acid Sequences

| | | SEQ |
|---|---|---|
| | Sequence | ID NO |
| CD3 HCDR1 | GYTMN | 1 |
| CD3 HCDR2 | LINPYKGVSTYNQKFKD | 2 |
| CD3 HCDR3 | SGYYGDSDWYFDV | 3 |
| CD3 LCDR1 | RASQDIRNYLN | 4 |
| CD3 LCDR2 | YTSRLES | 5 |
| CD3 LCDR3 | QQGNTLPWT | 6 |
| CD3 VH | EVQLVESGGGLVQPGGSLRLSCAASGYSFTGYTMNWVRQ | 7 |
| | APGKGLEWVALINPYKGVSTYNQKFKDRFTISVDKSKNTA | |
| | YLQMNSLRAEDTAVYYCARSGYYGDSDWYFDVWGQGTL | |
| | VTVSS | |
| CD3 VL | DIQMTQSPSSLSASVGDRVTITCRASQDIRNYLNWYQQKPG | 8 |
| | KAPKLLIYYTSRLESGVPSRFSGSGSGTDYTLTISSLQPEDFA | |
| | TYYCQQGNTLPWTFGQGTKVEIK | |
| WT1 HCDR1 | SYAIS | 9 |
| WT1 HCDR2 | GIIPIFGTANYAQKFQG | 10 |
| WT1 HCDR3 | SIELWWGGFDY | 11 |
| WT1 LCDR1 | RASQSISSWLA | 12 |
| WT1 LCDR2 | DASSLES | 13 |
| WT1 LCDR3 | QQYEDYTT | 14 |
| WT1 VH | QVQLVQSGAEVKKPGSSVKVSCKASGGTFSSYAISWVRQA | 15 |
| | PGQGLEWMGGIIPIFGTANYAQKFQGRVTITADKSTSTAYM | |
| | ELSSLRSEDTAVYYCARSIELWWGGFDYWGQGTTVTVSS | |
| WT1 VL | DIQMTQSPSTLSASVGDRVTITCRASQSISSWLAWYQQKPG | 16 |
| | KAPKLLIYDASSLESGVPSRFSGSGSGTEFTLTIGSLQPDDFA | |
| | TYYCQQYEDYTTFGQGTKVEIK | |
| WT1 VL- | DIQMTQSPSTLSASVGDRVTITCRASQSISSWLAWYQQKPG | 17 |
| CL(RK) | KAPKLLIYDASSLESGVPSRFSGSGSGTEFTLTIGSLQPDDFA | |
| | TYYCQQYEDYTTFGQGTKVEIKRTVAAPSVFIFPPSDRKLK | |
| | SGTASVVCLLNNFYPREAKVQWKVDNALQSGNSQESVTE | |
| | QDSKDSTYSLSSTLTLSKADYEKHKVYACEVTHQGLSSPVT | |
| | KSFNRGEC | |
| WT1 VH- | QVQLVQSGAEVKKPGSSVKVSCKASGGTFSSYAISWVRQA | 18 |
| CH1(EE)- | PGQGLEWMGGIIPIFGTANYAQKFQGRVTITADKSTSTAYM | |
| Fc(hole, | ELSSLRSEDTAVYYCARSIELWWGGFDYWGQGTTVTVSSA | |
| PGLALA) | STKGPSVFPLAPSSKSTSGGTAALGCLVEDYFPEPVTVSWN | |
| | SGALTSGVHTFPAVLQSSGLYSLSSVVTVPSSSLGTQTYICN | |
| | VNHKPSNTKVDEKVEPKSCDKTHTCPPCPAPEAAGGPSVFL | |
| | FPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVCTLPPSRDELTKNQ | |
| | VSLSCAVKGFYPSDIAVEWESNGQPENNYKTTPPVLDSDGS | |
| | FFLVSKLTVDKSRWQQGNVFSCSVMHEALHNHYTQKSLSL | |
| | SP | |
| CD3 VH-CL | EVQLVESGGGLVQPGGSLRLSCAASGYSFTGYTMNWVRQ | 19 |
| | APGKGLEWVALINPYKGVSTYNQKFKDRFTISVDKSKNTA | |
| | YLQMNSLRAEDTAVYYCARSGYYGDSDWYFDVWGQGTL | |
| | VTVSSASVAAPSVFIFPPSDEQLKSGTASVVCLLNNFYPREA | |
| | KVQWKVDNALQSGNSQESVTEQDSKDSTYSLSSTLTLSKA | |
| | DYEKHKVYACEVTHQGLSSPVTKSFNRGEC | |
| WT1 VH- | QVQLVQSGAEVKKPGSSVKVSCKASGGTFSSYAISWVRQA | 20 |
| CH1(EE)-CD3 | PGQGLEWMGGIIPIFGTANYAQKFQGRVTITADKSTSTAYM | |
| VL-CH1- | ELSSLRSEDTAVYYCARSIELWWGGFDYWGQGTTVTVSSA | |
| Fc(knob, | STKGPSVFPLAPSSKSTSGGTAALGCLVEDYFPEPVTVSWN | |
| PGLALA) | SGALTSGVHTFPAVLQSSGLYSLSSVVTVPSSSLGTQTYICN | |
| | VNHKPSNTKVDEKVEPKSCDGGGGSGGGGSDIQMTQSPSS | |
| | LSASVGDRVTITCRASQDIRNYLNWYQQKPGKAPKLLIYYT | |
| | SRLESGVPSRFSGSGSGTDYTLTISSLQPEDFATYYCQQGNT | |
| | LPWTFGQGTKVEIKSSASTKGPSVFPLAPSSKSTSGGTAALG | |
| | CLVKDYFPEPVTVSWNSGALTSGVHTFPAVLQSSGLYSLSS | |
| | VVTVPSSSLGTQTYICNVNHKPSNTKVDKKVEPKSCDKTH | |
| | TCPPCPAPEAAGGPSVFLFPPKPKDTLMISRTPEVTCVVVDV | |
| | SHEDPEVKFNWYVDGVEVHNAKTKPREEQYNSTYRVVSV | |
| | LTVLHQDWLNGKEYKCKVSNKALGAPIEKTISKAKGQPRE | |
| | PQVYTLPPCRDELTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSP | |
| Untargeted | EVQLLESGGGLVQPGGSLRLSCAASGFTFSSYAMSWVRQA | 21 |
| VH | PGKGLEWVSAISGSGGSTYYADSVKGRFTISRDNSKNTLYL | |
| | QMNSLRAEDTAVYYCAKGSGFDYWGQGTLVTVSS | |
| Untargeted VL | EIVLTQSPGTLSLSPGERATLSCRASQSVSSSYLAWYQQKP | 22 |
| | GQAPRLLIYGASSRATGIPDRFSGSGSGTDFTLTISRLEPEDF | |
| | AVYYCQQYGSSPLTFGQGTKVEIK | |
| Human WT1 | MGSDVRDLNALLPAVPSLGGGGGCALPVSGAAQWAPVLD | 23 |
| | FAPPGASAYGSLGGPAPPPAPPPPPPPPPHSFIKQEPSWGGA | |
| | EPHEEQCLSAFTVHFSGQFTGTAGACRYGPFGPPPPSQASS | |
| | GQARMFPNAPYLPSCLESQPAIRNQGYSTVTFDGTPSYGHT | |
| | PSHHAAQFPNHSFKHEDPMGQQGSLGEQQYSVPPPVYGCH | |
| | TPTDSCTGSQALLLRTPYSSDNLYQMTSQLECMTWNQMNL | |
| | GATLKGVAAGSSSSVKWTEGQSNHSTGYESDNHTTPILCG | |
| | AQYRIHTHGVFRGIQDVRRVPGVAPTLVRSASETSEKRPFM | |
| | CAYPGCNKRYFKLSHLQMHSRKHTGEKPYQCDFKDCERR | |
| | FSRSDQLKRHQRRHTGVKPFQCKTCQRKFSRSDHLKTHTR | |
| | THTGKTSEKPFSCRWPSCQKKFARSDELVRHHNMHQRNM | |
| | TKLQLAL | |
| VLD peptide | VLDFAPPGA | 24 |
| RMF peptide | RMFPNAPYL | 25 |
| HLA-A2 | GSHSMRYFFTSVSRPGRGEPRFIAVGYVDDTQFVRFDSDAA | 26 |
| | SQRMEPRAPWIEQEGPEYVVDGETRKVKAHSQTHRVDLGT | |
| | LRGYYNQSEAGSHTVQRMYGCDVGSDWRFLRGYHQYAY | |
| | DGKDYIALKEDLRSWTAADMAAQTTKHKWEAAHVAEQL | |
| | RAYLEGTCVEWLRRYLENGKETLQRTDAPKTHMTHHAVS | |
| | DHEATLRCWALSFYPAEITLTWQRDGEDQTQDTELVETRP | |
| | AGDGTFQKWAAVVVPSGQEQRYTCHVQHEGLPKPLTLRW | |
| | E | |
| Human CD3 | MQSGTHWRVLGLCLLSVGVWGQDGNEEMGGITQTPYKVS | 27 |
| | ISGTTVILTCPQYPGSEILWQHNDKNIGGDEDDKNIGSDEDH | |
| | LSLKEFSELEQSGYYVCYPRGSKPEDANFYLYLRARVCENC | |
| | MEMDVMSVATIVIVDICITGGLLLLVYYWSKNRKAKAKPV | |
| | TRGAGAGGRQRGQNKERPPPVPNPDYEPIRKGQRDLYSGL | |
| | NQRRI | |
| Cynomolgus | MQSGTRWRVLGLCLLSIGVWGQDGNEEMGSITQTPYQVSI | 28 |
| CD3 | SGTTVILTCSQHLGSEAQWQHNGKNKEDSGDRLFLPEFSE | |
| | MEQSGYYVCYPRGSNPEDASHHLYLKARVCENCMEMDV | |
| | MAVATIVIVDICITLGLLLLVYYWSKNRKAKAKPVTRGAG | |
| | AGGRQRGQNKERPPPVPNPDYEPIRKGQQDLYSGLNQRRI | |
| hIgG1 Fc | DKTHTCPPCPAPELLGGPSVFLFPPKPKDTLMISRTPEVTCV | 29 |
| region | VVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSRDELTKNQVSLTCLVKGFYPSDIAVEWE | |
| | SNGQPENNYKTTPPVLDSDGSFFLYSKLTVDKSRWQQGNV | |
| | FSCSVMHEALHNHYTQKSLSLSP | |

### BRIEF DESCRIPTION OF THE DRAWINGS

FIG. 1. Schematic illustration of the HLA-A2/WT1-targeted T-cell bispecific (TCB) antibody molecule used in the Examples (“WT1 TCB”). The molecule comprises a single antigen binding moiety for CD3, two antigen binding sites for HLA-A2/WT1, and an Fc domain.

FIG. 2. Lenalidomide enhances WT1 TCB-mediated cytotoxicity. (A) Representative example: specific lysis of primary AML cells on days 4, 7 and 13 after co-culture with healthy donor T cells. Upper left area of each panel: T-cells, lower right area of each panel: leukemic cells; with percentages given. (B) Summary: specific lysis of primary AML cells on day 4 of co-culture; median with interquartile range; Wilcoxon matched-pairs signed rank test; n=13.

FIG. 3. Cytokine levels in supernatants after 4 days of treatment with WT1 TCB and lenalidomide in co-cultures of primary AML cells with healthy donor T cells. (A, B, D, E, F) increased levels of pro-inflammatory cytokines upon combination with WT1-TCB ((A) interleukin (IL)-2, (B) TNF-a, (D) IFN-y, (E) IL-6, (F) IL-4), (C) decreased levels of anti-inflammatory IL-10; *: p<0.05, **: p<0.005, n.s.: not significant; Wilcoxon matched-pairs signed rank test; n=9.

FIG. 4. Phenotype of healthy donor CD3⁺ T cells in co-cultures with primary AML cells after treatment with WT1 TCB and lenalidomide. (a) Representative example of CD45RA and CCR7 expression analysis; (b) Percentages of Tₙₐᵢᵥₑ and T_{CM} after 7-10 days of treatment, median with interquartile range, Wilcoxon matched-pairs signed rank test, n=8.

### EXAMPLES

The following are examples of methods and compositions of the invention. It is understood that various other aspects may be practiced, given the general description provided above.

### Example 1

### Combination of WT1 TCB with Lenalidomide

### Materials and Methods

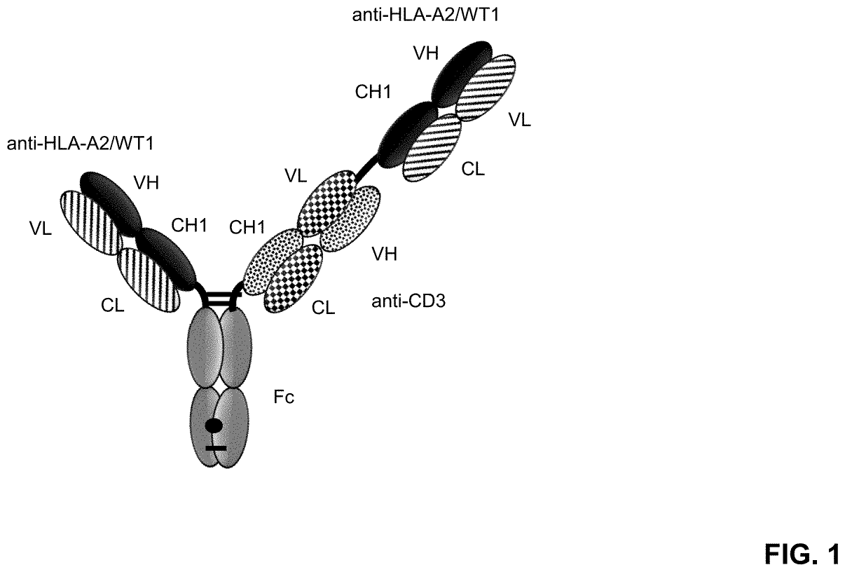

Ex vivo cytotoxicity assays using primary AML cells were performed in a-MEM medium supplemented with 10% fetal calf serum (FCS), 10% horse serum and 1% penicillin/streptomycin/glutamine. The medium was supplemented with recombinant human granulocyte-colony stimulating factor (rhG-CSF), interleukin (IL)-3 and thrombopoietin (TPO) (Peprotech, Hamburg, Germany) and 57.4 mM β-mercaptoethanol (Sigma-Aldrich, Munich, Germany). Primary AML cells were thawed and pre-cultivated on a feeder layer of irradiated murine MS5 stromal cells in a 6 well plate. After 3-4 days, primary AML cells were transferred onto a fresh feeder layer in a 96 well plate. WT1 TCB (SEQ ID NOs 9-16 (HLA-A2/WT1 CDRs and V-regions), 1-8 (CD3 CDRs and V-regions) and 17-20 (full heavy and light chains), molecular structure as in FIG. 1), was added at a concentration of 10 nM. Lenalidomide was added at a concentration of 10 μM. T cells from healthy donors were thawed and co-cultivated with the primary AML cells at a E:T ratio of 1:2 for 4 days. An untargeted TCB of similar structure (binding only CD3, but no tumor antigen, having SEQ ID NOs 21-22 as non-binding V-regions) was used as control.

Surface expression of CD33 (REA775), CD2 (REA972; both: Miltenyi, Heidelberg, Germany), CD69 (FN50), PD1 (29F.1Al2), TIM3 (F38-2E2), CD45RA (HI100), CCR7 (G43H7; all from: Biolegend, San Diego, USA) was assessed by flow cytometry (CytoFLEX S, Beckman Coulter Life Sciences, Krefeld, Germany). Cytokine concentrations in cell culture supernatants were quantified using the Human Th1/Th2 Cytokine Kit (BD Biosciences, Heidelberg, Germany).

### Results

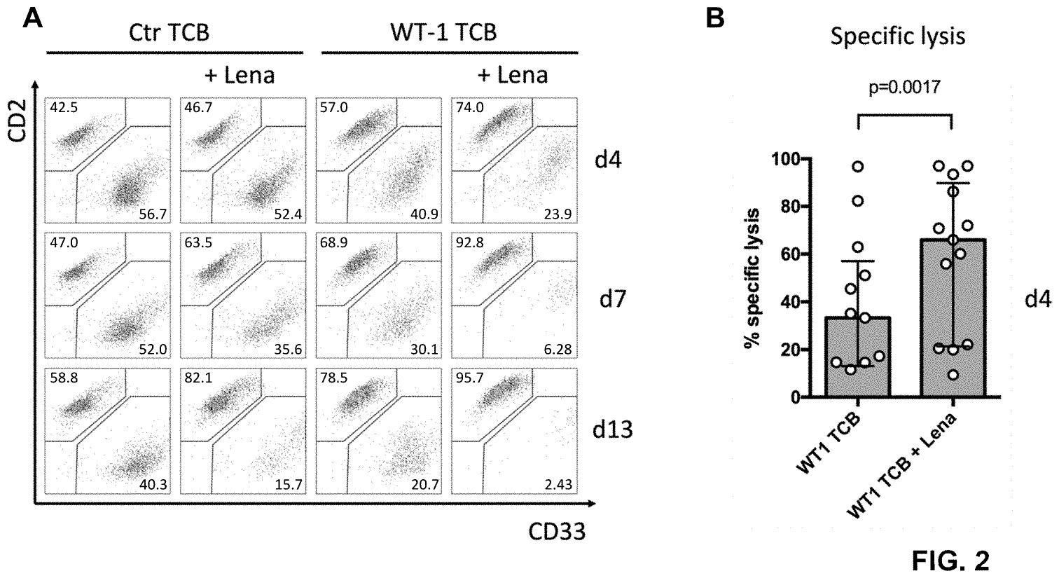

Combination of WT1 TCB with lenalidomide further enhanced WT1 TCB-mediated T cell cytotoxicity (mean specific lysis on day 3-4: 32±10% vs. 59±9%; p=0.0017; ±SEM; n=13), whereas the combination of lenalidomide with an untargeted control TCB did not result in a significant increase. See FIG. 2.

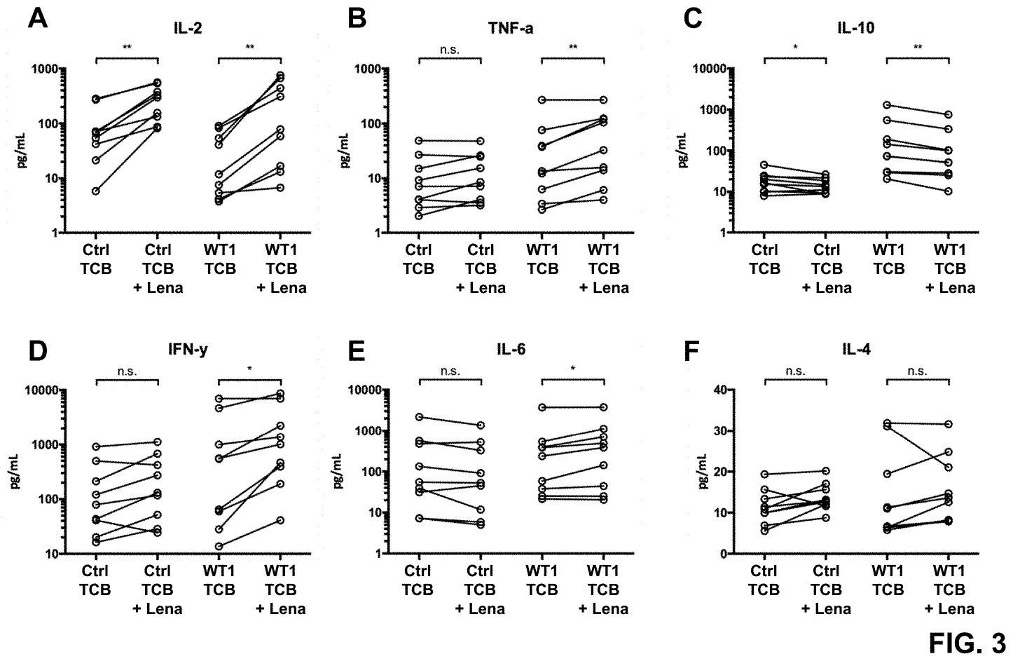

Combination of WT1 TCB with lenalidomide induces the secretion of pro-inflammatory cytokines and a reduction of the anti-inflammatory cytokine IL-10, whereas the combination of lenalidomide with an untargeted control TCB did not result in a significant change. See FIG. 3.

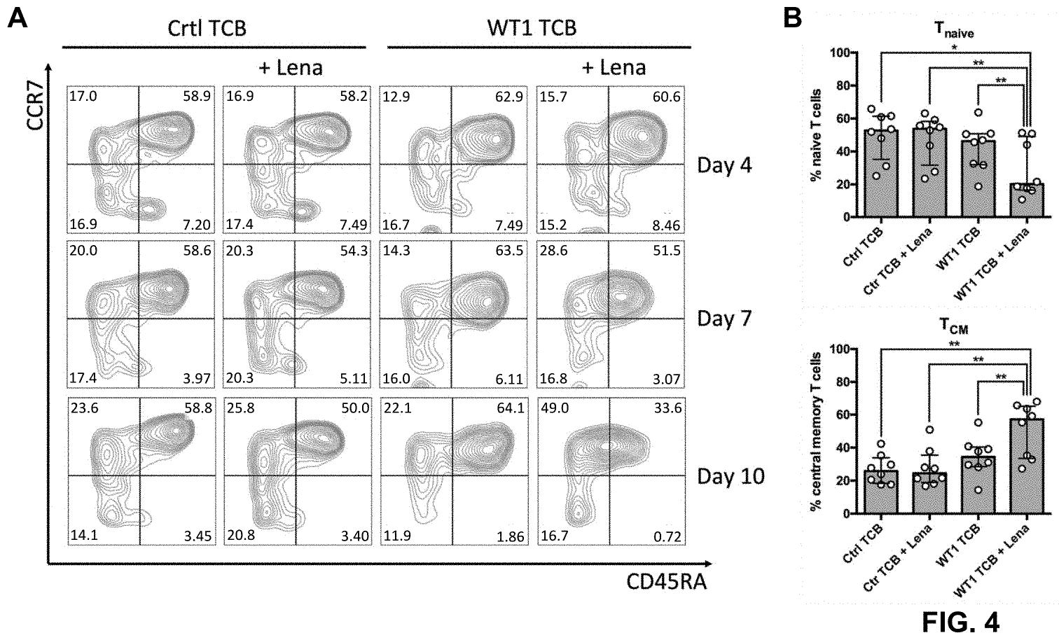

Combination of WT1-TCB with lenalidomide promotes the differentiation of naïve T cells towards the central memory (T_{CM}) phenotype, characterized by a downregulation of CD45RA, whereas the combination of lenalidomide with an untargeted control TCB did not affect differentiation. See FIG. 4.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A HLA-A2/WT1×CD3 bispecific antibody for use in the treatment of a cancer in an individual, wherein the treatment comprises administration of the HLA-A2/WT1×CD3 bispecific antibody in combination with lenalidomide.

2. Use of a HLA-A2/WT1×CD3 bispecific antibody in the manufacture of a medicament for the treatment of cancer in an individual, wherein the treatment comprises administration of the HLA-A2/WT1×CD3 bispecific antibody in combination with lenalidomide.

3. A method for treating cancer in an individual comprising administering to the individual a HLA-A2/WT1×CD3 bispecific antibody and lenalidomide.

4. A kit comprising a first medicament comprising a HLA-A2/WT1×CD3 bispecific antibody and a second medicament comprising lenalidomide, and optionally further comprising a package insert comprising instructions for administration of the first medicament in combination with the second medicament for treating cancer in an individual.

5. The HLA-A2/WT1×CD3 bispecific antibody for use, the use, the method or the kit of any one of the preceding claims, wherein the HLA-A2/WT1×CD3 bispecific antibody comprises
(i) a first antigen binding moiety that specifically binds to CD3 and comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6; and
(ii) a second antigen binding moiety that specifically binds to HLA-A2/WT1 and comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

6. The HLA-A2/WT1×CD3 bispecific antibody for use, the use, the method or the kit of any one of the preceding claims, wherein the HLA-A2/WT1×CD3 bispecific antibody comprises a third antigen binding moiety that specifically binds to HLA-A2/WT1 and/or an Fc domain composed of a first and a second subunit.

7. The HLA-A2/WT1×CD3 bispecific antibody for use, the use, the method or the kit of any one of the preceding claims, wherein the HLA-A2/WT1×CD3 bispecific antibody comprises
(i) a first antigen binding moiety that specifically binds to CD3, comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions of the Fab light chain and the Fab heavy chain are exchanged;
(ii) a second and a third antigen binding moiety that specifically bind to HLA-A2/WT1, comprising a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14, wherein the second and third antigen binding moiety are each a Fab molecule, particularly a conventional Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

8. The HLA-A2/WT1×CD3 bispecific antibody for use, the use, the method or the kit of any one of the preceding claims, wherein the first antigen binding moiety of the HLA-A2/WT1×CD3 bispecific antibody comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8, and/or the second and (where present) third antigen binding moiety of the HLA-A2/WT1×CD3 bispecific antibody comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

9. The HLA-A2/WT1×CD3 bispecific antibody for use, the use, the method or the kit of any one of the preceding claims, wherein the first antigen binding moiety of the HLA-A2/WT1×CD3 bispecific antibody is a crossover Fab molecule wherein the variable regions of the Fab light chain and the Fab heavy chain are exchanged, and wherein the second and (where present) third antigen binding moiety of the HLA-A2/WT1×CD3 bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

10. The HLA-A2/WT1×CD3 bispecific antibody for use, the use, the method or the kit of any one of the preceding claims, wherein the Fc domain of the HLA-A2/WT1×CD3 bispecific antibody comprises a modification promoting the association of the first and the second subunit of the Fc domain, and/or the Fc domain comprises one or more amino acid substitution that reduces binding to an Fc receptor and/or effector function.

11. The HLA-A2/WT1×CD3 bispecific antibody for use, the use, the method or the kit of any one of the preceding claims, wherein the cancer is a WT1-positive cancer.

12. The HLA-A2/WT1×CD3 bispecific antibody for use, the use, the method or the kit of any one of the preceding claims, wherein the cancer is acute myeloid leukemia (AML).

13. The invention as described hereinbefore.


SEQUENCE LISTING (29 sequences)

SEQ ID NO: 1 - protein, 5 aa, Artificial Sequence
  CD3 HCDR1
GYTMN

SEQ ID NO: 2 - protein, 17 aa, Artificial Sequence
  CD3 HCDR2
LINPYKGVSTYNQKFKD

SEQ ID NO: 3 - protein, 13 aa, Artificial Sequence
  CD3 HCDR3
SGYYGDSDWYFDV

SEQ ID NO: 4 - protein, 11 aa, Artificial Sequence
  CD3 LCDR1
RASQDIRNYLN

SEQ ID NO: 5 - protein, 7 aa, Artificial Sequence
  CD3 LCDR2
YTSRLES

SEQ ID NO: 6 - protein, 9 aa, Artificial Sequence
  CD3 LCDR3
QQGNTLPWT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 5 aa, Artificial Sequence
  WT1 HCDR1
SYAIS

SEQ ID NO: 10 - protein, 17 aa, Artificial Sequence
  WT1 HCDR2
GIIPIFGTANYAQKFQG

SEQ ID NO: 11 - protein, 11 aa, Artificial Sequence
  WT1 HCDR3
SIELWWGGFDY

SEQ ID NO: 12 - protein, 11 aa, Artificial Sequence
  WT1 LCDR1
RASQSISSWLA

SEQ ID NO: 13 - protein, 7 aa, Artificial Sequence
  WT1 LCDR2
DASSLES

SEQ ID NO: 14 - protein, 8 aa, Artificial Sequence
  WT1 LCDR3
QQYEDYTT

SEQ ID NO: 15 - protein, 120 aa, Artificial Sequence
  WT1 VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSS

SEQ ID NO: 16 - protein, 106 aa, Artificial Sequence
  WT1 VL
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIK

SEQ ID NO: 17 - protein, 213 aa, Artificial Sequence
  WT1 VL-CL(RK)
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 448 aa, Artificial Sequence
  WT1 VH-CH1(EE)-Fc(hole, PGLALA)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 229 aa, Artificial Sequence
  CD3 VH-CL
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 671 aa, Artificial Sequence
  WT1 VH-CH1(EE)-CD3 VL-CH1-Fc(knob, PGLALA)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 115 aa, Artificial Sequence
  Untargeted VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 22 - protein, 108 aa, Artificial Sequence
  Untargeted VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 23 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 24 - protein, 9 aa, Homo sapiens
VLDFAPPGA

SEQ ID NO: 25 - protein, 9 aa, Homo sapiens
RMFPNAPYL

SEQ ID NO: 26 - protein, 275 aa, Homo sapiens [sequence omitted]

SEQ ID NO: 27 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 28 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 29 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP